(19) United States
(12) Patent Application Publication
(10) Pub. No.: US 2023/0158030 A1
(43) Pub. Date: May 25, 2023

(54) METHODS AND COMPOSITION FOR TREATMENT OF COVID-19 ILLNESS REQUIRING HOSPITALIZATION

(71) Applicant: OHIO STATE INNOVATION FOUNDATION, Columbus, OH (US)

(72) Inventors: John C. BYRD, Columbus, OH (US); Jennifer WOYACH, Columbus, OH (US); Meixiao LONG, Columbus, OH (US); Zeinab EL BOGHDADLY, Columbus, OH (US)

(21) Appl. No.: 17/918,084

(22) PCT Filed: Apr. 10, 2021
(86) PCT No.: PCT/US2021/026750

§ 371 Date: Oct. 10, 2022

Related U.S. Application Data
(60) Provisional application No. 63/008,603, filed on Apr. 10, 2020.

(51) Int. Cl. A61K 31/519; A61K 45/06; A61P 31/14

(52) U.S. Cl. CPC A61K 31/519; A61K 45/06; A61P 31/14

(57) ABSTRACT

Disclosed are methods for the treatment of a coronavirus infection in a mammal comprising the step of administering to the mammal a therapeutically effective amount of ibrutinib. Also disclosed are disclosed methods of treating a subject for a clinical condition associated with hypercytokinemia, the method comprising: administering to the subject ibrutinib; wherein the subject is identified to have a higher level of at least one pro-inflammatory cytokine in a test sample obtained from the subject compared to a control sample or a reference value, to treat the subject for a mild, moderate, critical or severe form of a clinical condition associated with hypercytokinemia.This abstract is intended as a scanning tool for purposes of searching in the particular art and is not intended to be limiting of the present disclosure.

### CROSS-REFERENCE TO RELATED APPLICATIONS

This Application claims the benefit of U.S. Provisional Application No. 63/008,603, filed on Apr. 10, 2020, which is incorporated herein by reference in its entirety.

### BACKGROUND

The SARS-CoV-2 virus, a novel coronavirus originated from Wuhan China and represents a serious human to human pathogenic virus. SARS-COV-2 has a variable phenotype ranging from asymptomatic carrier state to rapidly progressive acute respiratory distress syndrome. The clinical syndrome manifested by SARS-CoV-2 is termed COVID-19. Both the infectiveness and pathogenic nature of this respiratory droplet transmitted RNA virus greatly exceeds that of influenza, likely as a consequence of no prior human exposure or available vaccine. SARS-CoV-2 has rapidly spread throughout the world and was recently by the WHO as a pandemic pathogen. Notably, SARS-COV-2 has common presentation of fever (89%), cough (68%), CT abnormalities (86%) and lympocytopenia (84%) with nausea (5%) and diarrhea (4%) being uncommon. Severe disease that requires admission occurs in 20% and a subset of these patients require ICU care (6%), intubation (2.3%) or die (1.4%). (1) The death rate from COVID-19 in different populations has been different from China (updated data, 2.4%) to Italy (7.2%). (2, 3). Death rate from COVID-19 increases proportionately with older age, being highest among older patients and also those who are immunocompromised or have other co-morbidities or lymphocytopenia (1, 3-5). Among hospitalized cancer patients with COVID-19 in China, a 39% frequency of mechanical ventilation or death occurred compared to 8% among patients without cancer (6). The systemic and pulmonary pathogenesis derived from clinical cases of ICU hospitalized covid-19 cases and autopsy series demonstrates increased levels of plasma inflammatory cytokines including TNF-alpha, IL-6, and IL-2, and IL-10 and neutrophilic infiltration, macrophages, monocytes, minimal lymphocytes (CD4+ T-cells predominately) and type 2 pneumocytes with EM viral particles. (7, 8) Other risk factors for severe covid-19 illness is the presence of neutrophilia, organ dysfunction (elevated LDH), coagulation abnormalities, thrombocytopenia, and lymphocytopenia suggesting both findings observed in hemophagocytic syndrome, and immune deficiency (1, 9, 10).

Covid-19 has rapidly evolved as a critical global health emergency lacking effective clinical treatments. These needs and other needs are satisfied by the present disclosure.

### SUMMARY

In accordance with the purpose (s) of the present disclosure, as embodied and broadly described herein, the disclosure, in one aspect, relates to methods for the treatment of a coronavirus infection in a mammal comprising the step of administering to the mammal a therapeutically effective amount of ibrutinib.

Disclosed are methods for the treatment of a disorder associated with coronavirus infection in a mammal comprising the step of administering to the mammal a therapeutically effective amount of at least one disclosed compound or pharmaceutically acceptable salt thereof.

Also disclosed are methods of treating a subject for a clinical condition associated with hypercytokinemia, the method comprising: administering to the subject ibrutinib; wherein the subject is identified to have a higher level of at least one pro-inflammatory cytokine in a test sample obtained from the subject compared to a control sample or a reference value, to treat the subject for a mild, moderate, critical or severe form of a clinical condition associated with hypercytokinemia.

Also disclosed are methods for inhibiting coronavirus infection in a mammal comprising the step of administering to the mammal a therapeutically effective amount of at least one disclosed compound or pharmaceutically acceptable salt thereof.

Also disclosed are methods for inhibiting coronavirus infection in at least one cell, comprising the step of contacting the cell with an effective amount of at least one disclosed compound or pharmaceutically acceptable salt thereof.

Also disclosed are uses of a disclosed compound, or a pharmaceutically acceptable salt thereof; a disclosed product of making, or a pharmaceutically acceptable salt thereof; or a disclosed pharmaceutical composition.

Also disclosed are uses of a disclosed compound, or a pharmaceutically acceptable salt thereof, in the manufacture of a medicament for the treatment of a disorder associated with a coronavirus infection in a mammal.

Also disclosed are methods for the manufacture of a medicament to decrease coronavirus infection in a mammal comprising combining at least one disclosed compound, or a pharmaceutically acceptable salt thereof with a pharmaceutically acceptable carrier or diluent.

Also disclosed are methods for the treatment of a disorder associated with hypercytokinemia in a mammal comprising the step of administering to the mammal a therapeutically effective amount of at least one disclosed compound or pharmaceutically acceptable salt thereof.

Also disclosed are methods for inhibiting hypercytokinemia in a mammal comprising the step of administering to the mammal a therapeutically effective amount of at least one disclosed compound or pharmaceutically acceptable salt thereof.

Also disclosed are methods for inhibiting hypercytokinemia in at least one cell, comprising the step of contacting the cell with an effective amount of at least one disclosed compound or pharmaceutically acceptable salt thereof.

Also disclosed are uses of a disclosed compound, or a pharmaceutically acceptable salt thereof; a disclosed product of making, or a pharmaceutically acceptable salt thereof; or a disclosed pharmaceutical composition.

Also disclosed are uses of a disclosed compound, or a pharmaceutically acceptable salt thereof, in the manufacture of a medicament for the treatment of a disorder associated with a hypercytokinemia in a mammal.

Also disclosed are methods for the manufacture of a medicament to decrease hypercytokinemia in a mammal comprising combining at least one disclosed compound, or a pharmaceutically acceptable salt thereof with a pharmaceutically acceptable carrier or diluent.

Also disclosed are methods for the treatment of a disorder associated with COVID-19 in a mammal comprising the step of administering to the mammal a therapeutically effective amount of at least one disclosed compound or pharmaceutically acceptable salt thereof.

Also disclosed are methods for inhibiting COVID-19 in a mammal comprising the step of administering to the mammal a therapeutically effective amount of at least one disclosed compound or pharmaceutically acceptable salt thereof.

Also disclosed are methods for inhibiting COVID-19 in at least one cell, comprising the step of contacting the cell with an effective amount of at least one disclosed compound or pharmaceutically acceptable salt thereof.

Also disclosed are uses of a disclosed compound, or a pharmaceutically acceptable salt thereof; a disclosed product of making, or a pharmaceutically acceptable salt thereof; or a disclosed pharmaceutical composition.

Also disclosed are uses of a disclosed compound, or a pharmaceutically acceptable salt thereof, in the manufacture of a medicament for the treatment of a disorder associated with a COVID-19 in a mammal.

Also disclosed are methods for the manufacture of a medicament to decrease COVID-19 in a mammal comprising combining at least one disclosed compound, or a pharmaceutically acceptable salt thereof with a pharmaceutically acceptable carrier or diluent.

In a further aspect, the present disclosure relates to kits comprising irbrutinib, or a pharmaceutically acceptable salt, hydrate, solvate, or polymorph thereof, and one or more of: (a) at least one agent known to decrease an inflammatory response; (b) at least one agent known to treat a disorder associated with cancer or cell hyperproliferation; (d) instructions for treating a disorder associated with hypercytokinemia; (e) instructions for treating a disorder associated with cancer; or (f) instructions for administering the compound in connection with treating COVID-19.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a therapeutic agent,” “a subject,” or “an infection,” including, but not limited to, two or more such therapeutic agents, subjects, or infections, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “ibrutinib” refers to a compound having a structure represented by a formula:

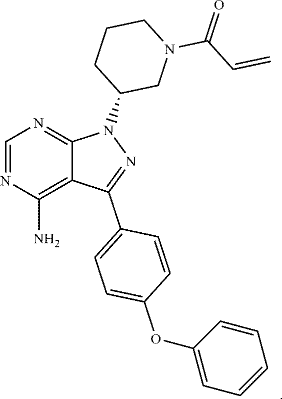

Ibrutinib is also the compound known to the skilled artisan as 1-[(3R)-3-[4-amino-3-(4-phenoxyphenyl)pyrazolo[3,4-d]pyrimidin-1-yl]piperidin-1-yl]prop-2-en-1-one having a molecular formula of C₂₅H₂₄N₆O₂. Other synonyms for ibrutinib are 936563-96-1; PCI-32765; IMBRUVICA®; PCI 32765; Ibrutinib (PCI-32765); PCI-32765 (Ibrutinib); UNII-1X70OSD4VX; CRA-032765; Pc-32765; 1X70OSD4VX; (R)-1-(3-(4-amino-3-(4-phenoxyphenyl)-1H-pyrazolo[3,4-d]pyrimidin-1-yl)piperidin-1-yl)prop-2-en-1-one; CHEBI:76612; PCI32765; 1-[(3R)-3-[4-Amino-3-(4-phenoxyphenyl)-1H-pyrazolo[3,4-d]pyrimidin-1-yl]-1-piperidinyl]-2-propen-1-one; A1-01649; 1-[(3R)-3-[4-amino-3-(4-phenoxyphenyl)pyrazolo[3,4-d]pyrimidin-1-yl]piperidin-1-yl]prop-2-en-1-one; 1-{(3R)-3-[4-amino-3-(4-phenoxyphenyl)-1H-pyrazolo[3,4-d]pyrimidin-1-yl]piperidin-1-yl}prop-2-en-1-one; 2-Propen-1-one, 1-((3R)-3-(4-amino-3-(4-phenoxyphenyl)-1H-pyrazolo(3,4-d)pyrimidin-1-yl)-1-piperidinyl)-; Ibrutinib [USAN:INN]; ibrutinibum; JNJ 02; Imbruvica (TN); 1-[(3R)-3-[4-Amino-3-(4-phenoxyphenyl)-1H-pyrazolo[3,4-d]pyrimidin-1-yl]piperidin-1-yl]prop-2-en-1-one; 2-Propen-1-one, 1-[(3R)-3-[4-amino-3-(4-phenoxyphenyl)-1H-pyrazolo[3,4-d]pyrimidin-1-yl]-1-piperidinyl]-; CRA 032765; PCI-32765-00; Ibrutinib (JAN/USAN); Ibrutinib/PCI-32765; Ibrutinib (PCI32765); Ibrutinib(PCI-32765); PCI-32765(lbrutinib); MLS006010041; SCHEMBL201859; GTPL6912; CHEMBL1873475; HSDB 8260; DTXSID60893450; EX-A066; XYFPWWZEPKGCCK-GOSISDBHSA-N; AMX10219; AOB87789; KS-000002KE; ABP000965; BDBM50357312; ZINC35328014; AKOS022185476; ACN-030256; DB09053; EBD2165770; EX-5960; KIN0000174; QC-4573; SB14736; NCGC00187912-01; NCGC00187912-02; NCGC00187912-03; NCGC00187912-12; AC-26942; HY-10997; SC-96633; SMR004701213; AB0008168; AX8254580; FT-0696693; SW218096-2; X7513; D10223; S-7810; J-523872; Q5984881; (R)-3-(4-amino-3-(4-phenoxyphenyl)-1H-pyrazolo[3,4-d]pyrimidin-1-yl)piperidin-1-ylprop-2-en-1-one; 1-((R)-3-(4-amino-3-(4-phenoxyphenyl)-1H-pyrazolo[3,4-d]pyrimidin-1-yl)piperidin-1-yl)prop-2-en-1-one; 1-[(3R)-3-[4-Amino-3-(4-phenoxyphenyl)-1H-pyrazolo[3,4-d]pyrimidin-1-yl]-1-piperidinyl]-2-propen-1-one1-[(3R)-3-[4-AMino-3-(4-phenoxyphenyl)pyrazolo [3, 4-d]pyriMidin-1-yl]; 1-[(3R)-3-[4-Amino-3-(4-phenoxyphenyl)-1H-pyrazolo[3,4-d]pyrimidin-1-yl]piperidin-1-yl]-2-propen-1-one; 1-[(3R)-3-[4-Amino-3-(4-phenoxyphenyl)pyrazolo[3, 4-d]pyrimidin-1-yl]piperidin-1 -yl]prop-2-en-1-one; IbrutinibCl-32765mbruvica(R)-1-(3-(4-amino-3-(4-phenoxyphenyl)-1H-pyrazolo[3,4-d]pyrimidin-1-yl)piperidin-1-yl)prop-2-en-1-one; and piperidin-1-yl]prop-2-en-1-one1-{3-[4-AMino-3-(4-phenoxy-phenyl)-pyrazolo[3,4-d]pyriMidin-1-yl]-piperidin-1-yl}-propenone.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians’ Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double-and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as covid-19. The effect can be therapeutic in terms of a partial or complete cure of a disease, condition, symptom or adverse effect attributed to the disease, disorder, or condition. The term “treatment” as used herein can include any treatment of covid-19 in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and juvenile subjects, whether male or female, are intended to be covered. In one aspect, the subject is a mammal. A patient refers to a subject afflicted with a disease or disorder. The term “patient” includes human and veterinary subjects. Aspects of the invention include methods of treating a subject suffering from a hypercytokinemia disease, such as COVID-19. The terms “subject,” “individual,” “host,” and “patient,” are used interchangeably herein and refer to any mammalian subject for whom diagnosis, treatment, or therapy is desired, such as humans.

As used herein, the term “severity of a disease” refers to the risk posed by the disease to a subject. Severity of a disease also dictates the extent of treatment necessary for appropriately treating the subject. For example, a disease can be mild, moderate, severe, or critical.

A mild disease may cause slight discomfort and may resolve without any treatment, for example, where a subject’s immune system neutralizes the disease. A moderate disease may cause more than slight discomfort and may require some treatment for the disease to resolve. A severe disease causes significant discomfort and would require extensive treatment. A critical disease is life threatening and would require hospitalization and extensive treatment, which may not be successful resulting in the subject’s death.

Acute respiratory distress syndrome (ARDS) is a respiratory failure caused by rapid and widespread inflammation in the lungs. In ARDS, fluid builds up in the alveoli thereby preventing the lungs from filling with enough air and reduced oxygen supply to the organs.

Sepsis is a potentially life-threatening condition caused by excessive inflammatory response to an infection. The excessive inflammatory response can trigger changes that can damage multiple organ systems.

Systemic inflammatory response syndrome (SIRS) a widespread inflammatory state affecting the entire body. Unlike sepsis, which is in response to an infection, SIRS can be in response to an infectious or noninfectious insult.

By hypercytokinemia disease is meant a disease condition that is characterized by the presence of hypercytokinemia (also known as a cytokine storm). As used herein, hypercytokinemia refers to a severe immune reaction in which the body releases too many cytokines into the blood too quickly. Cytokines play an important role in normal immune responses, but having a large amount of them released in the body all at once can be harmful. Hypercytokinemia can occur as a result of an infection, autoimmune condition, or other disease. It may also occur after treatment with some types of immunotherapy. Signs and symptoms include high fever, inflammation (redness and swelling), and severe fatigue and nausea. Sometimes, hypercytokinemia may be severe or life threatening and lead to multiple organ failure. In embodiments, hypercytokinemia diseases may be caused by a viral infection. In some instances, the hypercytokinemia disease that is treated by methods of the invention is COVID-19, which is caused by SARS-CoV-2 infection. In some instances, the disease, e.g., COVID-19, is severe or critical.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

Unless otherwise specified, temperatures referred to herein are based on atmospheric pressure (i.e. one atmosphere).

### Clinical Conditions and Cytokine Storm

Coronaviruses (CoVs), a large family of single-stranded RNA viruses, can infect a wide variety of animals, including humans, causing respiratory, enteric, hepatic and neurological diseases. [Yin, Y., Wunderink, R G, Respirology (2018) 23 (2): 130-37, citing Weiss, S R, Leibowitz, I L, Coronavirus pathogenesis. Adv. Virus Res. (2011) 81: 85-164]. Human coronaviruses, which were considered to be relatively harmless respiratory pathogens in the past, have now received worldwide attention as important pathogens in respiratory tract infection. As the largest known RNA viruses, CoVs are further divided into four genera: alpha-, beta-, gamma-and delta-coronavirus. In humans, CoVs cause mainly respiratory tract infections. Until November 2019, only six human coronaviruses (HCoVs) had been identified. These included the alpha-CoVs HCoV-NL63 and HCoV-229E and the beta-CoVs HCoV-OC43, HCoV-HKU1, severe acute respiratory syndrome-CoV (SARS-CoV), [Id., citing Drosten, C. et al. N. Engl. J. Med. (2003) 348: 1967-76] and Middle East respiratory syndrome-CoV (MERS-CoV) [Id., citing Zaki, A M et al. N. Engl. J. Med. (2012) 367: 1814-20].

Human-to-human transmission of SARS-CoV and MERS-CoV occurs mainly through nosocomial transmission. From 43.5-100% of MERS patients in individual outbreaks were linked to hospitals, [Id., citing Hunter, I C et al. Transmission of Middle East respiratory syndrome coronavirus infections in healthcare settings, Abu Dhabi. Emerg. Infect. Dis. (2016) 22: 647-56. Osong Public Health Res. Perspect. (2015) 6: 269-78], which was similar in SARS patients. [Anderson, R M et al. Philos. Trans. R. Soc. Lond. B. Biol. Sci. (2004) 359: 1091-105]. A study from the Republic of Korea revealed that index patients who transmitted to others had more non-isolated days in the hospital, body temperature of ≥38.5° C. and pulmonary infiltration of ≥3 lung zones. [Id., citing Kang, C K, et al. J. Korean Med. Sci. (2017) 32: 744-49]. Transmission between family members occurred in only 13-21% of MERS cases and 22-39% of SARS cases. [Id., citing Kang, C K, et al. J. Korean Med. Sci. (2017) 32: 744-49]. Another Korean study suggested that transmission of MERS from an asymptomatic patient is rare. [Id., citing Moon, S Y, Son, J S. Clin. Infect. Dis. (2017) 64: 1457-58]. In contrast to SARS-CoV and MERS-CoV, direct human-to-human transmission was not reported for the other four HCoVs. [Id., citing Woo, P C et al. Hong Kong Med. J. (2008) 15 (Suppl. 9): 46-47].

Current understanding of the pathogenesis of HCoVs infection is still limited. However, several significant differences in the pathogenesis exist among SARS-CoV, MERS-CoV and the other HCoVs.

Since the emergence of coronavirus disease 2019 (COVID-19) (formerly known as the 2019 novel coronavirus (2019-nCoV) in Wuhan, China in December 2019, which is caused by severe acute respiratory syndrome coronavirus 2 (SARS-CoV-2), more than 75,000 cases have been reported in 32 countries/regions, resulting in more than 2000 deaths worldwide. [Lai C-C, et al., Asymptomatic carrier state, acute respiratory disease, and pneumonia due to severe acute respiratory syndrome coronavirus 2 (SARS-CoV-2): Facts and myths. J. Microbiol. Immunol. Infect. (2020) doi.org/10.1016/j.imii.2020.02.012].

COVID-19 can present as an asymptomatic carrier state, acute respiratory disease, and pneumonia. Adults represent the population with the highest infection rate; however, neonates, children, and elderly patients can also be infected by SARS-CoV-2. In addition, nosocomial infection of hospitalized patients and healthcare workers, and viral transmission from asymptomatic carriers are possible. The most common finding on chest imaging among patients with pneumonia was ground-glass opacity with bilateral involvement. Severe cases are more likely to be older patients with underlying comorbidities compared to mild cases. Indeed, age and disease severity may be correlated with the outcomes of COVID-19. [Id].

Recognition of pathogens or foreign (non-self) substances within the body typically triggers an immune response. Generally, the immune response involves a production of cytokines. It is generally important that the production of cytokines is well regulated for maintaining homeostatic balance within the body. An imbalance in the production of cytokines, for example an excessive production of cytokines, in the body can cause significant damage to body tissues and organs.

A cytokine storm (which is also known as hypercytokinemia) is a significant immune response to pathogens that invade the body. The precise causation of cytokine storms within the body has not been definitively established. A possible causation of cytokine storms is an encounter, by the immune system, of a new and highly pathogenic pathogen. Cytokine storms are also associated with a number of infectious and non-infectious diseases, including influenza, adult respiratory distress syndrome (ARDS), and systemic inflammatory response syndrome (SIRS). It has been suggested that various viruses, e.g., coronavirus, the influenza A (H1N1) virus, and other viruses can trigger cytokine storms within the body.

During a cytokine storm, pro-inflammatory mediators include, but are not limited to pro-inflammatory cytokines, oxygen free radicals, pro-inflammatory enzymes, and coagulation factors, and are released by the immune cells of the body in response to an inflammatory stimulus, e.g., a bacterial or viral infection. Examples of immune cells include white blood cells (e.g., leukocytes), which includes phagocytes, macrophages, neutrophils, dendritic cells, mast cells, eosinophils, basophils, and natural killer cells, and lymphocytes. In some embodiments, the disclosed methods facilitate a decrease in release or secretion of the pro-inflammatory cytokine from the immune cell or inhibition of one or more pro-inflammatory enzyme.

Accumulating evidence has suggested that polypeptide mediators known as cytokines, including various lymphokines, interleukins, and chemokines, are important stimuli to collagen deposition in fibrosis. Released by resident tissue cells and recruited inflammatory cells, cytokines are thought to stimulate fibroblast proliferation and increased synthesis of extracellular matrix proteins, including collagen. For example, an early feature in the pathogenesis of idiopathic pulmonary fibrosis is alveolar epithelial and/or capillary cell injury. This promotes recruitment into the lung of circulating immune cells, such as monocytes, neutrophils, lymphocytes and eosinophils. These effector cells, together with resident lung cells, such as macrophages, alveolar epithelial and endothelial cells, then release cytokines, which stimulate target cells, typically fibroblasts, to replicate and synthesize increased amounts of collagen. Breakdown of extracellular matrix protein also may be inhibited, thereby contributing to the fibrotic process. (Coker and Laurent, Eur Respir J, 1998, 11: 1218-1221)

Numerous cytokines have been implicated in the pathogenesis of fibrosis, including, without limitation, transforming growth factor-β (TGF-β), tumor necrosis factor-α (TNF-α), platelet-derived growth factor (PDGF), insulin-like growth factor-1 (IGF-1), endothelin-1 (ET-1) and the interleukins, interleukin-1 (IL-1), interleukin-6 (IL-6), interleukin-8 (IL-8), and interleukin-17 (IL-17). Chemokine leukocyte chemoattractants, including the factor Regulated upon Activation in Normal T-cells, Expressed and Secreted (RANTES), are also thought to play an important role. Elevated levels of pro-inflammatory cytokines, such as Interleukin 8 (IL-8), as well as related downstream cell adhesion molecules (CAMs) such as intercellular adhesion molecule-1 (ICAM-1) and vascular cell adhesion molecule-1 (VCAM-1), matrix metalloproteinases such as matrix metalloproteinase-7 (MMP-7), and signaling molecules such as S100 calcium-binding protein A12 (S100A12, also known as calgranulin C), in the peripheral blood have been found to be associated with mortality, lung transplant-free survival, and disease progression in patients with IPF (Richards et al, Am J Respir Crit Care Med, 2012, 185: 67-76).

In various aspects, pro-inflammatory cytokines include, but are not limited to, interleukin-1 (IL1), interleukin-6 (IL6), interleukin-8 (IL8), interleukin-11 (IL-11), interleukin-12 (IL-12), interleukin-17 (IL-17), interleukin-17 (IL-17), interleukin-18 (IL-18), tumor necrosis factor-alpha (TNF-α), interferon-gamma (IFN-y), granulocyte-macrophage colony stimulating factor (GM-CSF), platelet-derived growth factor (PDGF), and transforming growth factor-beta (TGF-β). It will be understood by a person having ordinary skill in the art that references to pro-inflammatory cytokines in most embodiments of the present disclosure can refer any one or more of pro-inflammatory cytokines that are known in the art, including the above-listed examples of pro-inflammatory cytokines. As discussed above, cytokine storms have the potential to cause significant damage to body tissues and organs. For example, occurrence of cytokine storms in the lungs can cause an accumulation of fluids and immune cells, for example macrophages, in the lungs, and eventually block off the body’s airways thereby resulting in respiratory distress and even death.

The TGF-β family of proteins has a potent stimulatory effect on extracellular matrix deposition, and in fact has been used in constructing induced animal models of fibrosis through gene transfer. In vitro studies show that TGF-β1, secreted as a latent precursor, promotes fibroblast procollagen gene expression and protein synthesis. The data suggest that the other mammalian isoforms, TGF-β2 and TGF-β3, also stimulate human lung fibroblast collagen synthesis and reduce breakdown in vitro. In animal models of pulmonary fibrosis, enhanced TGF-β1 gene expression is temporally and spatially related to increased collagen gene expression and protein deposition. TGF-β1 antibodies reduce collagen deposition in murine bleomycin-induced lung fibrosis, and human fibrotic lung tissue shows enhanced TGF-β1 gene and protein expression.

As used herein throughout, “pro-inflammatory cytokine” and “proinflammatory cytokine” can be used interchangeably and refer a cytokine that has increased expression or activity linked to a bacterial or viral infection, including those cytokines associated with a cytokine storm.

As used herein throughout, “inflammatory mediator” and “pro-inflammatory mediator” can be used interchangeably and refer to a pro-inflammatory cytokine, an immune cell associated with an inflammatory response, and/or an pro-inflammatory enzyme. A pro-inflammatory enzyme includes, but is not limited to, cyclooxygenase, phosphodiesterase-4 (PDE-4) and inducible nitric oxide synthetase (iNOS). It will be understood by a person having ordinary skill in the art that references to pro-inflammatory cytokines or pro-inflammatory enzymes in most aspects of the present disclosure can refer any one or more of pro-inflammatory cytokines or pro-inflammatory enzyems, respectively, that are known in the art, including the above-listed examples of pro-inflammatory cytokines or pro-inflammatory enzymes.

It can be appreciated by one skilled in the art that “decrease” or “inhibition” or “modulation” of a referenced pro-inflammatory cytokine or pro-inflammatory enzyme includes mechanisms such as a direct decrease, inhibition, or modulation by acting directly on either protein or its expression pathway by acting on the mRNA or gene associated with the referenced pro-inflammatory cytokine or pro-inflammatory enzyme, or alternatively, indirectly decrease, inhibition, or modulation by acting on a protein, gene, or other biological that is in a signaling or control pathway that impacts the activity or expression of the referenced pro-inflammatory cytokine or pro-inflammatory enzyme.

### Pharmaceutical Compositions

In various aspects, the present disclosure relates to pharmaceutical compositions comprising a therapeutically effective amount of irbrutinib, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of irbrutinib, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of irbrutinib, a pharmaceutically acceptable salt, a hydrate thereof, a solvate thereof, a polymorph thereof, or a stereochemically isomeric form thereof. In a further aspect, irbrutinib, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of irbrutinib are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, irbrutinib comprising an acidic group or moiety, e.g., a carboxylic acid group, can be used to prepare a pharmaceutically acceptable salt. For example, irbrutinib may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, irbrutinib comprising a protonatable group or moiety, e.g., an amino group, can be used to prepare a pharmaceutically acceptable salt. For example, irbrutinib may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with an basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, irbrutinib, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise irbrutinib (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and irbrutinib, or a pharmaceutically acceptable salt thereof. In a further aspect, irbrutinib, or pharmaceutically acceptable salt thereof, can also be included in a pharmaceutical composition in combination with one or more other therapeutically active compounds. The instant pharmaceutical compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington’s Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Pharmaceutical compositions described herein comprising irbrutinib are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or y-cyclodextrins or their derivatives, in particular hydroxyalkyl substituted cyclodextrins, e.g. 2-hydroxypropyl-p-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer’s dextrose, dextrose and sodium chloride, lactated Ringer’s and fixed oils. Intravenous vehicles include fluid and nutrient replenishers, electrolyte replenishers such as those based on Ringer’s dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long-acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions containing irbrutinib, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the pharmaceutical compositions of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions requiring irbrutinib at an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. Irbrutinib can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific pharmaceutical composition employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for hypercytokinemia (e.g., treatment of one or more clinical conditions associated with a viral infection, e.g., coronavirus infection, and/or COVID-19) in mammals (e.g., humans) comprising combining irbrutinib with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed pharmaceutical compositions can be prepared from the irbrutinib. It is also understood that irbrutinib can be employed in the disclosed methods of using.

As already mentioned, the present disclosure relates to a pharmaceutical composition comprising a therapeutically effective amount of irbrutinib, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of irbrutinib.

As already mentioned, the present disclosure also relates to a pharmaceutical composition comprising a irbrutinib, a pharmaceutically acceptable salt, a hydrate thereof, a solvate thereof, a polymorph thereof, and one or more other drugs in the treatment, prevention, control, amelioration, or reduction of risk of diseases or conditions for irbrutinib or the other drugs may have utility as well as to the use of such a composition for the manufacture of a medicament. The present disclosure also relates to a combination of irbrutinib, a pharmaceutically acceptable salt, a hydrate thereof, a solvate thereof, a polymorph thereof, and a inflammatory inhibitor. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional anti-inflammation therapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

### Methods of Treating COVID-19

In various aspects, the present disclosure, as embodied and broadly described herein, the disclosure, in one aspect, relates to methods for the treatment of a coronavirus infection in a mammal comprising the step of administering to the mammal a therapeutically effective amount of ibrutinib. In a further aspect, the disclosed methods of treating coronavirus can further comprise administering an additional therapeutic agent. In a still further aspect, the disclosed methods for the treatment of a coronavirus infection in a mammal comprising the step of administering to the mammal a disclosed pharmaceutical composition comprising ibrutinib.

In a further aspect, the disclosed methods pertain relate to methods for the treatment of COVID-19 in a mammal comprising the step of administering to the mammal a therapeutically effective amount of ibrutinib. In various aspects, the disclosed methods of treating COVID-19 can further comprise administering an additional therapeutic agent. In a still further aspect, the disclosed methods for the treatment of COVID-19 in a mammal comprising the step of administering to the mammal a disclosed pharmaceutical composition comprising ibrutinib.

To date, there have been no studies published that serially follow immune response serially through patients with SARS-CoV-2 infection and COVID-19 syndrome. Several studies have demonstrated cross comparative studies of moderate versus severe COVID-19 syndrome where differential decline in CD4 and CD8 cells occurs with increasing features of both exhausted T-cells (increased PD1, LAG3, CTLA4, and decreased intracellular gamma-interferon) and inflammatory cytokines (11-13). These findings mimic other chronic virus and parasitic infections where inability CD4 T-cells to generate cytotoxic gamma-interferon and T-cell exhaustion contributes to failure of the host organism to clear viral infections. Application of a therapeutic that enhances Th1 polarization, diminishes T-cell exhaustion and decreases TNF and IL6 production by both T-cells and monocytes would represent a potential novel therapeutic approach to COVID-19 syndrome.

In contrast to immune suppression predicting higher infection rate and morbidity with covid-19, once this syndrome is active therapies directed at diminishing inflammation including corticosteroids, IL-6 neutralizing antibodies, and hydroxychloraquine or hydroxychloraquine with azithomycin have demonstrated antidotal benefit among ill patients. Patients recovering from covid-19 syndrome demonstrate evidence of humoral neutralizing antibodies (14, 15) and SARS-CoV-2 specific T-cells. Additionally, ill patients treated with plasma derived from patients who have developed COVID-19 and recovered with evidence of immune response have had evidence of clinical improvement (16). This provides evidence that strategies that can disrupt the acute inflammatory response associated with respiratory failure while preserving immune function could serve to effectively treat patients with covid-19.

Without wishing to be bound by a particular theory, it is believed that cancer based targeted therapies can be used to shut down inflammatory cytokine release while not compromising innate or adaptive immune response, and positively modulate the clinical course of covid-19. It has been observed that targeted agents directed at graft versus host disease while at the same time maintaining anti-leukemic activity in the setting of allogeneic stem cell transplant (17, 18). One such example of this is Ruxolitinib, a JAK2 inhibitor that was recently FDA approved for the use of acute graft versus host disease. JAK2 inhibitors also demonstrate profound reduction in cytokine production in myelofibrosis and other inflammatory diseases (19). Similarly, it has been previously demonstrated that ibrutinib, an irreversible inhibitor of BTK and ITK, has chronic graft versus host therapeutic potential in pre-clinical models and does not inhibit graft versus leukemia effect (20-22). As an irreversible ITK inhibitor, Ibrutinib has been shown to polarize CD4 cells to a Th1 phenotype in human CLL cells favoring gamma interferon production and in murine models enabling clearance of pathogens such as listeria monocytogenes and leishmania (23, 24). By enhancing T-cell function, ibrutinib has been shown to enhance other T-cell based therapies including TLR agonists (25), PD1 blockade (26), and CAR-T cells (27-29). The combination of CAR-T cells and ibrutinib in addition to being highly active was shown to also potentially diminish cytokine release syndrome as compared to an earlier report of administering CAR-T cells alone in CLL (30, 31). This was recapitulated experimentally in a murine model of CAR-T cell mediated cytokine release syndrome where ibrutinib improved survival by decreasing this complication while not abrogating T-cell expansion or killing (32). Finally, in work performed by the present inventors in CLL, it was demonstrated that ibrutinib via ITK inhibition diminishes both activation induced death and reversed features of immune exhaustion, a common pathway that T-cells become depleted in the setting of cancer or chronic viral infections.

Disclosed herein are methods for treatment of covid-19 in view of favorable immune modulating features of ibrutinib. In various aspects, the disclosed methods can enhance Th1 polarization, diminish exhaustion, and improve T-cell immune response, and accordingly provide a method of treatment toward SARS-COV-2.

Heretofore, ibrutinib has not been administered clinically to treat infectious disease or abrogate inflammatory toxicity mediated by immune hyeractivation in the lung. There is pre-clinical data implicating BTK activation in acute pulmonary injury and subsequent ARDS. TK activation has been demonstrated to be an essential component of lung injury induced by infectious pathogens, sepsis, and hemorrhagic lung injury that is mediated by excessive inflammatory macrophages and neutrophils (33-35). Genetic knock down of BTK impaired this lung injury (34, 35). Subsequent pharmacologic studies with ibrutinib have confirmed this. Specifically, ibrutinib in the setting of murine pneumococcal pneumonia has also been shown to negatively influence both monocyte and neutrophil influx into the lung and also alveolar macrophage activation, neutrophil influx, cytokine release and plasma leakage. Antibiotic mediated killing of bacteria was not impaired (36). A similar study with seasonal influenza A virus demonstrated ibrutinib administered intranasally to mice starting 72 h after lethal infection with influenza A diminished weight loss and led to improved overall survival (37). Additionally, ibrutinib treatment had a dramatic effect on morphological changes to the lungs including decreased alveolar hemorrhage, interstitial thickening, and the presence of alveolar exudate concomitant with diminished inflammatory mediators TNFalpha, IL-1beta, IL-6, KC, and MCP-1. This murine study suggests that ibrutinib may be an effective therapy for not only influenza-induced lung injury but for treating other viral pathogens mediating lung dysfunction via excessive inflammation (37). Collectively, these data support ibrutinib diminishing lung pathology mediated by the SARS-COV-2 virus and justify application of this approach for clinical investigation in patients with covid-19 syndrome.

Without wishing to be bound by a particular theory, it is believe that the disclosed methods can: 1) diminish the excessive innate immune inflammatory pulmonary response to SARS-COR-2 infection that causes need for intubation and often death; 2) helps to prevent/reverse the severe compromise of adaptive immune system by ameliorating lymphopenia, reducing exhaustion as measured by checkpoint molecule expression (PD-1/CTLA4 etc.), and preserving the Th1/Tc1 function, and can protect against HLH/MAS like symptoms, where deficiency in T cells or NK cells plays a key role; and 3) Enhance or at least not compromise viral clearance based upon data by our group with listeria and Leishmania major and others with influenza.

### Methods of Treating Hypercytokinemia

In various aspects, the present disclosure, as embodied and broadly described herein, the disclosure, in one aspect, relates to methods for the treatment of hypercytokinemia in a mammal comprising the step of administering to the mammal a therapeutically effective amount of ibrutinib. In a further aspect, the disclosed methods of treating hypercytokinemia can further comprise administering an additional therapeutic agent. In a still further aspect, the disclosed methods for the treatment of hypercytokinemia in a mammal comprising the step of administering to the mammal a disclosed pharmaceutical composition comprising ibrutinib.

As discussed above, hypercytokinemia is a potentially fatal immune reaction and involves a positive feedback loop between cytokines and immune cells, which causes in the body highly elevated levels of various cytokines. Hypercytokinemia is also referenced as “cytokine storm.” Hypercytokinemia typically involves increased concentration of cytokines, such as interferons, interleukins, chemokines, colony-stimulating factors, and tumor necrosis factors. Such immune dysregulation can be an underlying factor in mortality resulting from many infections.

In certain embodiments, the disease involving hypercytokinemia further comprises an overproduction of immune cells and/or pro-inflammatory cytokines into the lungs of the subject. Hypercytokinemia may result in excessive inflammatory response in the lungs, which typically occurs in the infections in the lungs or other organs. Such excessive inflammatory response in the lung includes infiltration into the lungs by immune cells as well as excessive secretion of proinflammatory cytokines in the lungs, which in turn attracts more immune cells into the lungs. Thus, hypercytokinemia can also involve positive-feedback loop between activated cells and released cytokines.

Described herein are methods of treating hypercytokinemia. In various aspects, the disclosed methods comprise administering irbrutinib to a subject in need thereof. In a further aspect, the subject can have hypercytokinemia. In a still further aspect, the subject can have a coronavirus infection, e.g., a coronavirus infection associated with a clinical diagnosis of covid-19. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Kits

In a further aspect, the present disclosure relates to kits comprising irbrutinib, or a pharmaceutically acceptable salt, hydrate, solvate, or polymorph thereof, and one or more of: (a) at least one agent known to decrease an inflammatory response; (b) at least one agent known to treat a disorder associated with cancer or cell hyperproliferation; (d) instructions for treating a disorder associated with hypercytokinemia; (e) instructions for treating a disorder associated with cancer; or (f) instructions for administering the compound in connection with treating COVID-19.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

### Methods of Determining Hypercytokinemia Severity

As summarized above, certain embodiments of the present disclosure provides methods of assessing the severity of a disease involving hypercytokinemia in a subject, the method comprising: (a) determining the level of pro-inflammatory cytokine in a test sample obtained from the subject, and (b) assessing the severity of the disease based on the determined level of pro-inflammatory cytokine in the test sample. Assessing the severity of the disease may involve comparing the determined level of pro-inflammatory cytokine in the test sample with the level of pro-inflammatory cytokine in a control sample or a reference value.

A control sample can be obtained from one or more of the following: an individual belonging to the same species as the subject and not having the disease; an individual belonging to the same species as the subject and known to have the disease with known level of severity, for example, asymptomatic, mild, moderate, severe, or critical; or the subject prior to having the disease. If a control sample is obtained from a healthy individual and the level of pro-inflammatory cytokine in the test sample is significantly higher than that of the control sample, particularly, higher than that of the control sample by: between 2 fold and 200 fold; between 10 fold and 150 fold, between 20 fold and 100 fold, or between 40 fold and 60 fold, the extent of the increase in the level of pro-inflammatory cytokine in the test sample could be used to assess the severity of the disease in the subject.

For example, \an increase of between at least about 6 fold and at least about 20 fold in the pro-inflammatory cytokine level in a test plasma sample compared to a control sample could indicate that the subject has a severe form of COVID-19; whereas, an increase of between at least about 60 fold and at least about 300 fold in the pro-inflammatory cytokine level in a test plasma sample compared to a control sample could indicate that the subject has a critical form of COVID-19.

For a particular disease involving hypercytokinemia, a person of ordinary skill in the art can determine the difference in the pro-inflammatory cytokine level in a control sample and in a test sample that indicates different levels of severity of the disease.

The control sample and the test sample can be obtained from the same type of an organ or tissue. The organ or tissue can be brain, eyes, pineal gland, pituitary gland, thyroid gland, parathyroid glands, thorax, heart, lung, esophagus, thymus gland, pleura, adrenal glands, appendix, gall bladder, urinary bladder, large intestine, small intestine, kidneys, liver, pancreas, spleen, stoma, ovaries, uterus, or skin. The control sample and the test sample can also be obtained from the same type of a body fluid. The body fluid can be aqueous humor, vitreous humor, bile, blood, cerebrospinal fluid, chyle, endolymph, perilymph, lymph, mucus, pericardial fluid, peritoneal fluid, pleural fluid, pus, rheum, saliva, sputum, synovial fluid, blood, serum or plasma.

A reference value corresponding to the level of pro-inflammatory cytokine may indicate the level of pro-inflammatory cytokine associated with severity of the disease, such as mild, moderate, severe, or critical form of a disease. As such, a reference value corresponding to level of pro-inflammatory cytokine may represent the level of pro-inflammatory cytokine in a subject who has mild, moderate, severe, or critical form of a disease.

For example, when the disease is COVID-19, the reference value can be: 200 pg/ml, 250 pg/ml; 300 pg/ml, 350 pg/ml or 400 pg/ml (which would indicate the absence of disease); or about: 1 ng/ml, 2 ng/ml, 3 ng/ml, 4 ng/ml, 5 ng/ml, 6 ng/ml, 7 ng/ml, 8 ng/ml, 9 ng/ml, 10 ng/ml (which would indicate severe form of COVID-19); or: 15 ng/ml, 20 ng/ml, 25 ng/ml, 30 ng/ml, 35 ng/ml, 40 ng/ml, 45 ng/ml, 50 ng/ml, 55 ng/ml, 60 ng/ml (which could indicate critical form of COVID-19). Therefore, if the pro-inflammatory cytokine level in a test sample from a subject is 50 ng/ml, one could assess that the subject is suffering from or could develop a critical form of COVID-19. On the other hand, if pro-inflammatory cytokine level in a test sample from a subject is 8 ng/ml, one could assess that the subject is suffering from or could develop a severe form of COVID-19. Further, if pro-inflammatory cytokine level in a test sample from a subject is 200 pg/ml, one could assess that the subject does not have COVID-19 or has asymptomatic or mild form of COVID-19.

A person of ordinary skill in the art can obtain appropriate reference values based on the disease being examined and such information can be obtained from the well-known sources in the relevant art or generated based on the testing conducted on a case-by-case basis.

If the disease is COVID-19, the disease can be asymptomatic, mild, moderate, severe, or critical. An asymptomatic form of COVID-19 does not show any symptoms in the subject. A mild form of COVID-19 may show mild form of one or more of: tiredness, fever, cough, breathlessness after moderate exercise, sore throat, muscle ache, headache, and diarrhea. Mild form of COVID-19 may not require management of symptoms. A moderate form of COVID-19 may show moderate form of one or more of: tiredness, fever, cough, breathlessness after slight activity, sore throat, muscle ache, headache, and diarrhea. Moderate form of COVID-19 may require managing the symptoms. A severe form of COVID-19 may show of one or more of: severe tiredness, high fever, cough, breathlessness even at rest, painful breathing, loss of appetite, loss of thirst, sore throat, muscle ache, headache, diarrhea, and confusion. Severe form of COVID-19 would typically require significant intervention for managing symptoms, such as: pneumonia, hypoxemic respiratory failure, ARDS, sepsis, septic shock, cardiomyopathy, arrhythmia, acute kidney injury, and complications from prolonged hospitalization including secondary bacterial infections, thromboembolism, gastrointestinal bleeding, and critical illness polyneuropathy/myopathy.

A critical form of COVID-19 may show of one or more of: severe tiredness, high fever, cough, breathlessness even at rest, painful breathing, loss of appetite, loss of thirst, sore throat, muscle ache, headache, diarrhea, confusion, severe pneumonia, ARDS, sepsis, organ failure, coma, and death. Critical form of COVID-19 requires hospitalization for managing symptoms such as: pneumonia, ARDS, sepsis, septic shock, cardiomyopathy, arrhythmia, acute kidney injury, and complications from prolonged hospitalization including secondary bacterial infections, thromboembolism, gastrointestinal bleeding, and critical illness polyneuropathy/myopathy. Ventilator assisted breathing may be required.

A disease involving hypercytokinemia can be an inflammatory disease or an infection. An inflammatory disease can be an autoimmune disease, graft rejection, multiple sclerosis, pancreatitis, or multiple organ dysfunction syndrome. A disease involving hypercytokinemia can also be an infection, such as a viral, bacterial, fungal, or parasitic infection. Additional examples of infections that can cause a disease involving hypercytokinemia are known in the art and severity of such diseases can be assessed according to the methods disclosed herein. A bacterial infection can comprise bacteremia, bacterial sepsis, pneumonia, cellulitis, meningitis, erysipelas, infective endocarditis, necrotizing fasciitis, prostatitis, pseudomembranous colitis, pyelonephritis, or septic arthritis. A bacterial infection can be caused by a Streptococcus spp., Staphylococcus spp., Salmonella spp., Pseudomonas spp., Clostridium spp., Vibrio spp., Mycobacterium spp. or Haemophilus spp. Additional examples of bacteria that can cause a disease involving hypercytokinemia are known in the art and severity of such bacterial infections can be assessed according to the methods disclosed herein. A viral infection can be caused by a coronavirus, influenza virus, Epstein-Barr virus, Human Immunodeficiency Virus, Ebola virus, retrovirus, or variola virus. The coronavirus can be severe acute respiratory syndrome coronavirus 2 (SARS-CoV-2). Additional examples of viruses that can cause a disease involving hypercytokinemia are known in the art and severity of such viral infections can be assessed according to the methods disclosed herein. A disease involving hypercytokinemia can also comprise ARDS, sepsis, SIRS, or toxic shock syndrome.

Any convenient method of determining pro-inflammatory cytokine in a same may be employed, where various methods of determining pro-inflammatory cytokine in a sample are known in the art and can be used in the methods disclosed herein. Certain such methods include flow cytometry, mass spectrometry, protein array analysis, Western blot analysis, enzyme-linked immunosorbent assay (ELISA), or radio-immune assay (RIA).

In certain embodiments, determining the level of pro-inflammatory cytokine is performed by flow cytometry. Flow cytometry is a methodology using multi-parameter data for identifying and distinguishing between different particle (e.g., bead) types i.e., particles that vary from one another in terms of label (wavelength, intensity), size, etc., in a fluid medium. In flow cytometrically analyzing a sample, an aliquot of the sample is first introduced into the flow path of the flow cytometer. When in the flow path, the particles in the sample are passed substantially one at a time through one or more sensing regions, where each of the cells is exposed separately and individually to a source of light at a single wavelength (or in some instances two or more distinct sources of light) and measurements of cellular parameters, e.g., light scatter parameters, and/or marker parameters, e.g., fluorescent emissions, as desired, are separately recorded for each cell. The data recorded for each cell is analyzed in real time or stored in a data storage and analysis means, such as a computer, for later analysis, as desired.

In flow cytometry-based methods, particles, e.g., beads, are passed, in suspension, substantially one at a time in a flow path through one or more sensing regions where in each region each cell is illuminated by an energy source. The energy source may include an illuminator that emits light of a single wavelength, such as that provided by a laser (e.g., He/Ne or argon) or a mercury arc lamp or an LED with appropriate filters. For example, light at 488 nm may be used as a wavelength of emission in a flow cytometer having a single sensing region. For flow cytometers that emit light at two distinct wavelengths, additional wavelengths of emission light may be employed, where specific wavelengths of interest include, but are not limited to: 405 nm, 535 nm, 561 nm, 635 nm, 642 nm, and the like. Following excitation of a labeled specific binding member bound to a polypeptide by an energy source, the excited label emits fluorescence and the quantitative level of the polypeptide on each cell may be detected, by one or more fluorescence detectors, as it passes through the one or more sensing regions.

In flow cytometry, in addition to detecting fluorescent light emitted from particles labeled with fluorescent markers, detectors, e.g., light collectors, such as photomultiplier tubes (or “PMT”), an avalanche photodiode (APD), etc., are also used to record light that mediated by, e.g., emitted by a label on, the particle. Flow cytometers may further include one or more electrical detectors. In certain embodiments, an electrical detector may be employed for detecting a disturbance caused by a particle passing through an electrical field propagated across an aperture in the path of the particles. Such flow cytometers having electrical detectors will contain a corresponding electrical energy emitting source that propagates an electrical field across the flow path or an aperture through which cells are directed. Any convenient electrical field and/or combination of fields with appropriate detector(s) may be used for the detection and/or measurement of particles passing through the field including but not limited to, e.g., a direct current electrical field, alternating current electrical field, a radio-frequency field, and the like.

Flow cytometers further include data acquisition, analysis and recording means, such as a computer, wherein multiple data channels record data from each detector for each cell as it passes through the sensing region. The purpose of the analysis system is to classify and count cells wherein each cell presents itself as a set of digitized parameter values and to accumulate data for the sample as a whole.

The flow cytometry can comprise a bead-based assay, such as a sandwich protocol for bead based assay. In one embodiment, determining the level of pro-inflammatory cytokine by flow cytometry comprises contacting the sample with a bead comprising an antibody that specifically binds to pro-inflammatory cytokine, washing the bead and contacting the washed bead with a fluorescently labeled secondary antibody that specifically binds to pro-inflammatory cytokine, washing the bead and detecting the presence of pro-inflammatory cytokine on the bead by detecting by flow cytometry the label on the bead.

In a specific embodiment, determining the level of pro-inflammatory cytokine by flow cytometry comprises contacting the sample with a bead comprising an antibody that specifically binds to pro-inflammatory cytokine, washing the bead and contacting the washed bead with a biotinylated secondary antibody that specifically binds to pro-inflammatory cytokine, washing the bead and contacting the washed bead with a fluorescently labeled streptavidin, and detecting the presence of pro-inflammatory cytokine on the bead by detecting by flow cytometry the fluorescent label on the bead. In some instances, the assay employed is BioLegend’s LEGENDplex™ bead-based immunoassay (BioLegend, San Diego, Calif.).

The fluorescent label used to detect the bead can be selected from a large number of dyes that are commercially available from a variety of sources, such as Molecular Probes (Eugene, Oreg.) and Exciton (Dayton, Ohio). Examples of fluorophores of interest include, but are not limited to, 4-acetamido-4′-isothiocyanatostilbene-2,2′disulfonic acid; acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumarin 151); cyanine and derivatives such as cyanosine, Cy3, Cy5, Cy5.5, and Cy7; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; or combinations thereof. Other fluorophores or combinations thereof known to those skilled in the art may also be used, for example those available from Molecular Probes (Eugene, Oreg.) and Exciton (Dayton, Ohio).

Also of interest as specific binding members are those nucleic acid dyes or stains containing intrinsic fluorescence including those that specifically label DNA. Dyes and stains that are specific for DNA (or preferentially bind double stranded polynucleotides in contrast to single-stranded polynucleotides) and therefore may be employed as non-specific stains include, but are not limited to: Hoechst 33342 (2′-(4-Ethoxyphenyl)-5-(4-methyl-1-piperazinyl)-1H,1′H-2,5′-bibenzimidazole trihydrochloride) and Hoechst 33258 (4-[6-(4-Methyl-1-piperazinyl)-1′,3′-dihydro-1H,2′H-2,5′-bibenzimidazol-2′-ylidene]-2,5-cyclohexadien-1-one trihydrochloride) and others of the Hoechst series; SYTO 40, SYTO 11, 12, 13, 14, 15, 16, 20, 21, 22, 23, 24, 25 (green); SYTO 17, 59 (red), DAPI, DRAQ5™ (an anthraquinone dye with high affinity for double stranded DNA), YOYO-1, propidium iodide, YO-PRO-3, TO-PRO-3, YOYO-3 and TOTO-3, SYTOX Green, SYTOX, methyl green, acridine homodimer, 7-aminoactinomycin D, 9-amino-6-chloro-2-methoxyactridine.

In certain embodiments, determining the level of pro-inflammatory cytokine is performed by ELISA. ELISA can be direct ELISA, indirect ELISA, competitive ELISA, or sandwich ELISA. Various methods of conducting ELISA assay are known in the art and can be used in the methods disclosed herein. An example of such an assay is the Quantikine® ELISA Human pro-inflammatory cytokine Immunoassay (RnD Systems, Inc.).

Additional methods of determining plasma pro-inflammatory cytokine level are described in Published U.S. Pat. Application Publication No. 20120238460, the disclosure of which is herein incorporated by reference.

As such, certain embodiments of the disclosure provide a method comprising: assaying the level of pro-inflammatory cytokine in a sample obtained from a subject suffering from a disease involving hypercytokinemia. Such methods can further comprise assaying the level of pro-inflammatory cytokine in a control sample and/or obtaining one or more reference values corresponding to the level of pro-inflammatory cytokine. The details of control samples and reference values discussed above are applicable to the methods of assaying pro-inflammatory cytokine. A sample for assaying the level of pro-inflammatory cytokine can be obtained from a subject suffering from various diseases discussed above. Moreover, various methods discussed above could be used for assaying the level of pro-inflammatory cytokine in a sample.

In certain embodiments, the methods of assaying the level of pro-inflammatory cytokine in a sample obtained from a subject further comprises treating the subject for the disease. Certain such embodiments comprise treating a subject by administering to the subject an inhibitor of pro-inflammatory cytokine or an inhibitor of CCR5, e.g., as described above. In some embodiments, such disease is a severe or critical form of COVID-19. Various therapeutic methods discussed above, particularly, various inhibitors of pro-inflammatory cytokine or CCR5 discussed above can be used in the methods of treating a subject discussed herein.

### Devices for Determining Hypercytokinemia Severity

Further embodiments of the disclosure provide a device configured to indicate whether the level of pro-inflammatory cytokine in a sample is above or below a predetermined threshold.

In certain such embodiments, the device is a flow cytometer. The flow cytometer can comprise a signal processing unit that is configured to indicate whether the level of pro-inflammatory cytokine in a sample is above or below a predetermined threshold. Such signal processing unit can comprise a physical computer-readable medium comprising instructions that, when executed, indicate whether the level of pro-inflammatory cytokine in a sample is above or below a predetermined threshold.

The predetermined threshold for pro-inflammatory cytokine level can be about: 200 pg/ml, 250 pg/ml, 300 pg/ml, 350 pg/ml, 400 pg/ml, 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 25 ng/ml, 30 ng/ml, 35 ng/ml, 40 ng/ml, 45 ng/ml, 50 ng/ml, 55 ng/ml, or 60 ng/ml.

In certain such embodiments, the device comprises an internal control that provides a signal corresponding to the pro-inflammatory cytokine level at the predetermined threshold. Accordingly, a signal intensity higher than that of the internal control would indicate that the level of pro-inflammatory cytokine in the tested sample is higher than the predetermined threshold, whereas, a signal intensity lower than that of the internal control would indicate that the level of pro-inflammatory cytokine in the tested sample is lower than the predetermined threshold.

In some cases, the device is provided with a separate container containing a control having pro-inflammatory cytokine at a concentration of the predetermined threshold.

### REFERENCES

References are cited herein throughout using the format of reference number (s) enclosed by parentheses corresponding to one or more of the following numbered references. For example, citation of references numbers 1 and 2 immediately herein below would be indicated in the disclosure as (1, 2).

1. Guan WJ, Ni ZY, Hu Y, Liang WH, Ou CQ, He JX, Liu L, Shan H, Lei CL, Hui DSC, et al. Clinical Characteristics of Coronavirus Disease 2019 in China. The New England journal of medicine. 2020.

2. Wu Z, and McGoogan JM. Characteristics of and Important Lessons From the Coronavirus Disease 2019 (COVID-19) Outbreak in China: Summary of a Report of 72314 Cases From the Chinese Center for Disease Control and Prevention. Jama. 2020.

3. Livingston E, and Bucher K. Coronavirus Disease 2019 (COVID-19) in Italy. Jama. 2020.

4. Wang D, Hu B, Hu C, Zhu F, Liu X, Zhang J, Wang B, Xiang H, Cheng Z, Xiong Y, et al. Clinical Characteristics of 138 Hospitalized Patients With 2019 Novel Coronavirus-Infected Pneumonia in Wuhan, China. Jama. 2020.

5. Onder G, Rezza G, and Brusaferro S. Case-Fatality Rate and Characteristics of Patients Dying in Relation to COVID-19 in Italy. Jama. 2020.

6. Liang W, Guan W, Chen R, Wang W, Li J, Xu K, Li C, Ai Q, Lu W, Liang H, et al. Cancer patients in SARS-CoV-2 infection: a nationwide analysis in China. The Lancet Oncology. 2020;21 (3):335-7.

7. Huang C, Wang Y, Li X, Ren L, Zhao J, Hu Y, Zhang L, Fan G, Xu J, Gu X, et al. Clinical features of patients infected with 2019 novel coronavirus in Wuhan, China. Lancet. 2020;395 (10223):497-506.

8. Yao XH, Li TY, He ZC, Ping YF, Liu HW, Yu SC, Mou HM, Wang LH, Zhang HR, Fu WJ, et al. [A pathological report of three COVID-19 cases by minimally invasive autopsies]. Zhonghua bing li xue za zhi = Chinese journal of pathology. 2020;49 (0):E009.

9. Wu C, Chen X, Cai Y, Xia J, Zhou X, Xu S, Huang H, Zhang L, Zhou X, Du C, et al. Risk Factors Associated With Acute Respiratory Distress Syndrome and Death in Patients With Coronavirus Disease 2019 Pneumonia in Wuhan, China. JAMA internal medicine. 2020.

10. Lippi G, Plebani M, and Michael Henry B. Thrombocytopenia is associated with severe coronavirus disease 2019 (COVID-19) infections: A meta-analysis. Clinica chimica acta; international journal of clinical chemistry. 2020.

11. Chen G, Wu D, Guo W, Cao Y, Huang D, Wang H, Wang T, Zhang X, Chen H, Yu H, et al. Clinical and immunologic features in severe and moderate Coronavirus Disease 2019. The Journal of clinical investigation. 2020.

12. Zheng HY, Zhang M, Yang CX, Zhang N, Wang XC, Yang XP, Dong XQ, and Zheng YT. Elevated exhaustion levels and reduced functional diversity of T cells in peripheral blood may predict severe progression in COVID-19 patients. Cellular & molecular immunology. 2020.

13. Qin C, Zhou L, Hu Z, Zhang S, Yang S, Tao Y, Xie C, Ma K, Shang K, Wang W, et al. Dysregulation of immune response in patients with COVID-19 in Wuhan, China. Clinical infectious diseases : an official publication of the Infectious Diseases Society of America. 2020.

14. Walls AC, Park YJ, Tortorici MA, Wall A, McGuire AT, and Veesler D. Structure, Function, and Antigenicity of the SARS-CoV-2 Spike Glycoprotein. Cell. 2020.

15. Xiao DAT, Gao DC, and Zhang DS. Profile of Specific Antibodies to SARS-CoV-2: The First Report. The Journal of infection. 2020.

16. Shen C, Wang Z, Zhao F, Yang Y, Li J, Yuan J, Wang F, Li D, Yang M, Xing L, et al. Treatment of 5 Critically Ill Patients With COVID-19 With Convalescent Plasma. Jama. 2020.

17. Carniti C, Gimondi S, Vendramin A, Recordati C, Confalonieri D, Bermema A, Corradini P, and Mariotti J. Pharmacologic Inhibition of JAK1/JAK2 Signaling Reduces Experimental Murine Acute GVHD While Preserving GVT Effects. Clinical cancer research : an official journal of the American Association for Cancer Research. 2015;21 (16):3740-9.

18. Betts BC, Veerapathran A, Pidala J, Yang H, Horna P, Walton K, Cubitt CL, Gunawan S, Lawrence HR, Lawrence NJ, et al. Targeting Aurora kinase A and JAK2 prevents GVHD while maintaining Treg and antitumor CTL function. Science translational medicine. 2017;9 (372).

19. Kleppe M, Kwak M, Koppikar P, Riester M, Keller M, Bastian L, Hricik T, Bhagwat N, McKenney AS, Papalexi E, et al. JAK-STAT pathway activation in malignant and nonmalignant cells contributes to MPN pathogenesis and therapeutic response. Cancer discovery. 2015;5 (3):316-31.

20. Dubovsky JA, Flynn R, Du J, Harrington BK, Zhong Y, Kaffenberger B, Yang C, Towns WH, Lehman A, Johnson AJ, et al. Ibrutinib treatment ameliorates murine chronic graft-versus-host disease. The Journal of clinical investigation. 2014;124 (11):4867-76.

21. Schutt SD, Fu J, Nguyen H, Bastian D, Heinrichs J, Wu Y, Liu C, McDonald DG, Pidala J, and Yu XZ. Inhibition of BTK and ITK with Ibrutinib Is Effective in the Prevention of Chronic Graft-versus-Host Disease in Mice. PloS one. 2015;10 (9):e0137641.

22. Miklos D, Cutler CS, Arora M, Waller EK, Jagasia M, Pusic I, Flowers ME, Logan AC, Nakamura R, Blazar BR, et al. Ibrutinib for chronic graft-versus-host disease after failure of prior therapy. Blood. 2017;130 (21):2243-50.

23. Dubovsky JA, Beckwith KA, Natarajan G, Woyach JA, Jaglowski S, Zhong Y, Hessler JD, Liu TM, Chang BY, Larkin KM, et al. Ibrutinib is an irreversible molecular inhibitor of ITK driving a Th1-selective pressure in T lymphocytes. Blood. 2013;122 (15):2539-49.

24. Varikuti S, Volpedo G, Saljoughian N, Hamza OM, Halsey G, Ryan NM, Sedmak BE, Seidler GR, Papenfuss TL, Oghumu S, et al. The Potent ITK/BTK Inhibitor Ibrutinib Is Effective for the Treatment of Experimental Visceral Leishmaniasis Caused by Leishmania donovani. The Journal of infectious diseases. 2019;219 (4):599-608.

25. Sagiv-Barfi I, Kohrt HE, Burckhardt L, Czerwinski DK, and Levy R. Ibrutinib enhances the antitumor immune response induced by intratumoral injection of a TLR9 ligand in mouse lymphoma. Blood. 2015;125 (13):2079-86.

26. Sagiv-Barfi I, Kohrt HE, Czerwinski DK, Ng PP, Chang BY, and Levy R. Therapeutic antitumor immunity by checkpoint blockade is enhanced by ibrutinib, an inhibitor of both BTK and ITK. Proceedings of the National Academy of Sciences of the United States of America. 2015;112 (9):E966-72.

27. Fraietta JA, Beckwith KA, Patel PR, Ruella M, Zheng Z, Barrett DM, Lacey SF, Melenhorst JJ, McGettigan SE, Cook DR, et al. Ibrutinib enhances chimeric antigen receptor T-cell engraftment and efficacy in leukemia. Blood. 2016;127 (9):1117-27.

28. Ruella M, Kenderian SS, Shestova O, Fraietta JA, Qayyum S, Zhang Q, Maus MV, Liu X, Nunez-Cruz S, Klichinsky M, et al. The Addition of the BTK Inhibitor Ibrutinib to Anti-CD19 Chimeric Antigen Receptor T Cells (CART19) Improves Responses against Mantle Cell Lymphoma. Clinical cancer research : an official journal of the American Association for Cancer Research. 2016;22 (11):2684-96.

29. Qin JS, Johnstone TG, Baturevych A, Hause RJ, Ragan SP, Clouser CR, Jones JC, Ponce R, Krejsa CM, Salmon RA, et al. Antitumor Potency of an Anti-CD19 Chimeric Antigen Receptor T-Cell Therapy, Lisocabtagene Maraleucel in Combination With Ibrutinib or Acalabrutinib. Journal of immunotherapy. 2020.

30. GauthierJ, Hirayama AV, Purushe J, Hay KA, Lymp J, Li D, Yeung C, Sheih A, Pender BS, Hawkins RM, et al. Feasibility and efficacy of CD19-targeted CAR-T cells with concurrent ibrutinib for CLL after ibrutinib failure. Blood. 2020.

31. Turtle CJ, Hay KA, Hanafi LA, Li D, Cherian S, Chen X, Wood B, Lozanski A, Byrd JC, Heimfeld S, et al. Durable Molecular Remissions in Chronic Lymphocytic Leukemia Treated With CD19-Specific Chimeric Antigen Receptor-Modified T Cells After Failure of Ibrutinib. Journal of clinical oncology: official journal of the American Society of Clinical Oncology. 2017;35 (26):3010-20.

32. Ruella M, Kenderian SS, Shestova O, Klichinsky M, Melenhorst JJ, Wasik MA, Lacey SF, June CH, and Gill S. Kinase inhibitor ibrutinib to prevent cytokine-release syndrome after anti-CD19 chimeric antigen receptor T cells for B-cell neoplasms. Leukemia. 2017;31 (1):246-8.

33. Krupa A, Fudala R, Florence JM, Tucker T, Allen TC, Standiford TJ, Luchowski R, Fol M, Rahman M, Gryczynski Z, et al. Bruton’s tyrosine kinase mediates FcgammaRlla/Toll-like receptor-4 receptor crosstalk in human neutrophils. American journal of respiratory cell and molecular biology. 2013;48 (2):240-9.

34. Krupa A, Fol M, Rahman M, Stokes KY, Florence JM, Leskov IL, Khoretonenko MV, Matthay MA, Liu KD, Calfee CS, et al. Silencing Bruton’s tyrosine kinase in alveolar neutrophils protects mice from LPS/immune complex-induced acute lung injury. American journal of physiology Lung cellular and molecular physiology. 2014;307 (6):L435-48.

35. Zhou P, Ma B, Xu S, Zhang S, Tang H, Zhu S, Xiao S, Ben D, and Xia Z. Knockdown of Burton’s tyrosine kinase confers potent protection against sepsis-induced acute lung injury. Cell biochemistry and biophysics. 2014;70 (2):1265-75.

36. de Porto AP, Liu Z, de Beer R, Florquin S, de Boer OJ, Hendriks RW, van der Poll T, and de Vos AF. Btk inhibitor ibrutinib reduces inflammatory myeloid cell responses in the lung during murine pneumococcal pneumonia. Molecular medicine. 2019;25 (1):3.

37. Florence JM, Krupa A, Booshehri LM, Davis SA, Matthay MA, and Kurdowska AK. Inhibiting Bruton’s tyrosine kinase rescues mice from lethal influenza-induced acute lung injury. American journal of physiology Lung cellular and molecular physiology. 2018;315 (1):L52-L8.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in °C or is at ambient temperature, and pressure is at or near atmospheric.

### Prospective Clinical Trial

The prospective clinical trial will determine if ibrutinib effectively diminishes the high observed frequency of need for mechanical ventilation or death by administering ibrutinib therapy for hospitalized covid-19 patients with a history of cancer. Based on data obtained in this prospective clinical trial, if positive, then an expanded prospective clinical trial can be carried out broadening the eligibility to non-cancer patients. Details of the proposal are as described herein below.

Primary Objective: To determine the ability of ibrutinib administration within 24 hours of hospital admission in cancer patients can diminish the need for mechanical ventilation or death as compared to untreated patients

Secondary Objectives: 1) To examine baseline features and outcome treated on this therapeutic study; 2)To determine time to becoming febrile free (24 hour time period) among patients treated with ibrutinib versus control; 3) To determine the proportion of patients with viral clearance at time of hospital discharge and thereafter among patients treated with ibrutinib versus control; 4) To determine 5) To determine the time to discharge for patients receiving ibrutinib versus control treatment 6) To examine immune cell subsets for absolute number, activation, exhaustion markers, and presence of maturation arrest (NK cells) over time among patients treated with ibrutinib versus control; 5) To examine T-cell repertoire over time among patients treated with ibrutinib versus control; and 6) To determine serial change cytokines including IL6, IL1B, TNF-alpha, IL-10 serum levels over time among patients treated with ibrutinib versus control.

Eligibility: 1) Known prior diagnosis of cancer (solid or hematologic) or precursor cancer (MGUS or MBL) that is associated with immune suppression; 2) COVID-19 infection requiring hospitalization; 3) Creatinine < 2 x ULN; Bilirubin < 2 x ULN (unless Gilbert’s disease); ALT/AST < 5 x ULN; platelet count > 30;

Exclusion: 1) Medical need full medical anti-coagulation; 2) myocardial infarct or stroke within 6 months; 3) active need for mechanical ventilation; 4) Known bleeding disorders (eg, von Willebrand’s disease) or hemophilia; 5) History of stroke or intracranial hemorrhage within 6 months prior to enrollment; 6) pregnancy.

Study Design: 2:1 randomized phase 2 study comparing active intervention with ibrutinib + standard treatment versus standard treatment alone. Randomization will be stratified based upon age > 60 years. A 6 patient run in for safety and feasibility would occur prior to initiating the randomized design. If 2 or more patients had severe toxicity within 1 week attributable to ibrutinib than the study would not move forward. This study will be performed at OSU initially but if initial success is noted, expansion to one or more sites may occur to rapidly complete accrual.

Treatment: Ibrutinib 420 mg daily until 2 successive negative SARS-COV-2 PCR tests + standard of care versus standard of care alone. This dose of ibrutinib is chosen based upon the extensive CLL experience of the 420 mg and our own data demonstrating significant T-cell modulation when administered in this manner.

Correlative studies: These would include 1) baseline geriatric assessment, immune senescence markers measured by DNA epigenetic age, clonal hematopoiesis and other SNP biomarkers of interest to OSUWMC investigators and outcome; 2) serial immune and cytokine monitoring using CLIA approved cytokine, immunophenotypic analysis, and Cytof testing, 3) serial SARS-COV-2 viral clearance studies; 4) Detailed study of T-cell repertoire and expansion studies of SARS-COV-2 specific T-cells for future investigation among patients recovering from therapy.

Statistical Design: The expected rate of intubation or death in cancer patients is 38% from one retrospective study whereas with all patients without cancer it is 8% among different hospitals in China as a consequence of COVID-19 syndrome. A 6 patient run in of ibrutinib monotherapy will occur to assure feasibility and early safety. If severe adverse events attributable to ibrutinib are not noted in 1 or less patients during the first week, we will proceed to the randomized phase 2 study. This randomized phase 2 study would seek to test for a difference in the percentage of cancer patients who require intubation or die, assuming a 40% event rate in those receiving control treatment deemed best by the medical team and a 15% event rate in those receiving ibrutinib matching the expected frequency of infection/death. With an initial sample size estimate equal to 56, there is 80% power to detect this difference in proportions using a one-sided Fisher’s exact test and constraining the type I error to 0.20. The study would use a 2:1 randomization ratio to minimize patients receiving control therapy to enable more rapid enrollment to an active therapy for COVID-19. An interim analysis after approximately 10-12 patients are enrolled on the ibrutinib arm will be planned to allow for decisions regarding futility or sample size re-estimation based on conditional power calculations, since there is uncertainty in the design assumptions. Adverse events will be regularly monitored to identify patterns of adverse events that may outweigh benefit and subsequently influence decision-making. For all biomarkers, descriptive statistics (means, medians, standard deviations, interquartile range) and graphical displays will be used to characterize central tendency and variability over time. Values will be log transformed as appropriate to reflect biologic plausibility. We will evaluate for statistical trends over time among the biologic measurements using mixed effects models with multiple measurements per patient.

Implementation: The implementation of a clinical trial such as this in the rapidly evolving SARS-CoV-2 virus pandemic will require rapid review by OSUCCC scientific review committee, OSU IRB, and an FDA investigator-initiated IND. Drug supply would be required along with support for management of the trial. The prospective clinical trial can be rapidly initiated and use the CLL clinical trial coordinator group at OSU, who have extensive experience with ibrutinib use and toxicity. The control population with serial monitoring will also enable other investigators at the to study other immune changes and potentially direct alternative therapies such as PD1 blockade toward patients with this disease if exhausted T-cell phenotype is observed. All of these studies could be performed by a small group of laboratory members.

It should be emphasized that the above-described embodiments of the present disclosure are merely possible examples of implementations set forth for a clear understanding of the principles of the disclosure. Many variations and modifications may be made to the above-described embodiment (s) without departing substantially from the spirit and principles of the disclosure. All such modifications and variations are intended to be included herein within the scope of this disclosure and protected by the following claims.

1. A method of treating a subject for a coronavirus infection, the method comprising: administering to the subject a therapeutically effective amount of ibrutinib, thereby treating the subject for the coronavirus infection.

2. The method of claim 1, wherein the subject is a mammal.

3. The method of claim 2, wherein the mammal is a human.

4. The method of claim 1, wherein the subject has been diagnosed with a coronavirus infection prior to the administering step.

5. The method of claim 1, further comprising the step of identifying a mammal in need of treatment of a coronavirus infection.

6. The method of claim 1, wherein the coronavirus infection is associated with a clinical diagnosis of COVID-19 infection.

7. The method of claim 6, wherein the COVID-19 infection is mild, severe, or critical.

8. A method of treating a subject for a clinical condition associated with hypercytokinemia, the method comprising: administering to the subject ibrutinib; wherein the subject is identified to have a higher level of at least one pro-inflammatory cytokine in a test sample obtained from the subject compared to a control sample or a reference value, to treat the subject for a mild, moderate, critical or severe form of a clinical condition associated with hypercytokinemia.

9. The method of claim 8, wherein the disease involving hypercytokinemia further comprises an overproduction of immune cells and pro-inflammatory cytokines into the lungs of the subject.

10. The method of claim 8, wherein the disease is an inflammatory disease or an infection.

11. The method of claim 10, wherein the infection is a viral, bacterial, fungal, or parasitic infection.

12. The method of claim 11, wherein the infection is a bacterial infection.

13. The method of claim 12, wherein the bacterial infection comprises bacteremia, bacterial sepsis, pneumonia, cellulitis, meningitis, erysipelas, infective endocarditis, necrotizing fasciitis, prostatitis, pseudomembranous colitis, pyelonephritis, or septic arthritis.

14. The method of claim 11, wherein the infection is a viral infection.

15. The method of claim 14, wherein the viral infection is caused by a coronavirus, influenza virus, Epstein-Barr virus, Human Immunodeficiency Virus, Ebola virus, retrovirus, or variola virus.

16. The method of claim 15, wherein the viral infection is caused by a coronavirus.

17. The method of claim 16, wherein the coronavirus is severe acute respiratory syndrome coronavirus 2 (SARS-CoV-2).

18. A kit comprising ibrutinib, or a pharmaceutically acceptable salt thereof; and one or more of:
a. at least one agent known to treat a virus infection;
b. at least one agent known to treat inflammation; or
c. instructions for treating covid-19.

19. The kit of claim 18, wherein the ibrutinib and the at least one agent are co-formulated.

20. The kit of claim 18, wherein the ibrutinib and the at least one agent are co-packaged.
